(19) United States
(12) Patent Application Publication
(10) Pub. No.: US 2016/0317213 A1
(43) Pub. Date: Nov. 3, 2016

(54) UROLOGICAL INSTRUMENT

(71) Applicant: OLYMPUS WINTER & IBE GMBH, Hamburg (DE)

(72) Inventors: Michael WOLTER, Hamburg (DE); Janna Brit LANGHOLZ, Neustadt (DE)

(73) Assignee: OLYMPUS WINTER & IBE GMBH, Hamburg (DE)

(21) Appl. No.: 15/108,643

(22) PCT Filed: Mar. 4, 2015
(86) PCT No.: PCT/EP2015/000483

§ 371 Date: Jun. 28, 2016

(30) Foreign Application Priority Data
Mar. 26, 2014 (DE) 10 2014 004 290.1

(51) Int. Cl. A61B 18/14; G01K 13/00; G01K 7/16

(52) U.S. Cl. CPC A61B 18/149; G01K 7/16; G01K 13/002; A61B 2018/00517

(57) ABSTRACT

A urological instrument having a working electrode, to which a high frequency can be applied in a bladder filled with liquid, and having a temperature sensor arranged at a fixed distance therefrom, is characterized in that the temperature sensor has two measuring electrodes which are arranged at a fixed distance relative to one another, can be brought into liquid contact, and are connected to a resistance measuring device.

The invention relates to a urological instrument according to the preamble of claim 1.

A generic urological instrument is disclosed in DE 10 10 2011 121 A1. The urological instrument here is a resectoscope with a working electrode to which a high frequency is applied and which is operated in the liquid located in the bladder. With the working electrode, body tissue is removed by cutting or vaporizing, for example in the reduction of the hypertrophic prostate.

In modern high-performance instruments, in order to speed up the operation, very high levels of power are used which are emitted by the working electrode to the surroundings, and thus this leads to a very high temperature in the liquid of the bladder. These temperatures can have a tissue-damaging effect. Therefore, measures are necessary for monitoring the temperature in the bladder.

For this purpose, the known design provides a temperature sensor with thermochromic material which is arranged in the vicinity of the working electrode and which can be read optically by means of the optical unit for monitoring the operation site.

This known design has a number of disadvantages, in particular the laborious measuring method which can be carried out by the surgeon by observation of the sensor and comparison with a color table.

U.S. Pat. No. 6,197,021 B1 shows a surgical temperature-generating working electrode, the temperature of which is determined in the immediate vicinity with electrical sensors, such as for example thermocouples or thermistors. Although in this case precise electrical measuring methods are used, such electrically operating systems in a liquid environment, such as for example in a bladder, are extremely susceptible to malfunction.

There is also a further significant disadvantage of all known generic temperature sensors, namely the substantially localized measurement which leads to considerable agitation effects in liquid. In the vicinity of the working electrode which heats up considerably, large local temperature differences are produced in the liquid in the bladder and are agitated by flows, for example the irrigating flow. The measurement result is then more dependent upon the flow rate and chance than upon the actual temperature at the relevant location.

The object of the present invention is to provide a urological instrument which enables a more precise information to be obtained about the temperature in the bladder. This object is achieved by the features of claim 1.

The starting point for the invention is the recognition that the electrical conductivity of a liquid is highly dependent upon the temperature thereof. Therefore, by means of a resistance measuring device, the urological instrument according to the invention determines the electrical resistance of the liquid located in the bladder between two measuring electrodes and thus the conductivity of the liquid. In this case the measuring electrodes are kept at a fixed distance apart and in contact with the liquid. Thus the resistance of the liquid between the measuring electrodes can be determined, the inverse of the resistance, namely the electrical conductivity, being substantially linearly dependent upon the temperature of the liquid in the relevant measurement area. Thus simple and precise temperature measurements are possible. There are no electrical problems with the liquid; in fact, the contact of the measuring electrodes with the liquid is actually necessary for the measurement. With the invention there is no need to fear the agitation effect of the known temperature measuring devices, since the invention provides for measurement to be performed between two measuring electrodes, that is to say, in a measurement volume in which localized temperature differences are averaged out over the measurement volume. This also results in very stable measurement.

In order to maintain the precise spacing between the measuring electrodes, according to claim 2 these electrodes are advantageously fastened to an insulating spacer which ensures the spacing.

The resistance measuring device could be arranged adjacent to the measuring electrodes, but according to claim 3 it is advantageously arranged at a distance and connected by means of measuring leads. In particular, it can be set up in dry conditions away from the bladder.

Advantageously according to claim 4, the urological instrument is designed in such a way that the measuring electrode is fixedly connected to the working electrode. Thus, if the surgeon permanently changes the location of the working electrode, the measuring electrodes always remain in the vicinity of the temperature-generating elements.

In the case of a urological instrument with support arms holding the working electrode, it is advantageous according to claim 5 to arrange a measuring electrode on each support arm. Thus by means of the support arms, which serve as spacers according to claim 2, the measuring electrodes are kept at a precise distance in the vicinity of the working electrode and always move therewith.

The invention is illustrated schematically by way of example in the drawings, in which:

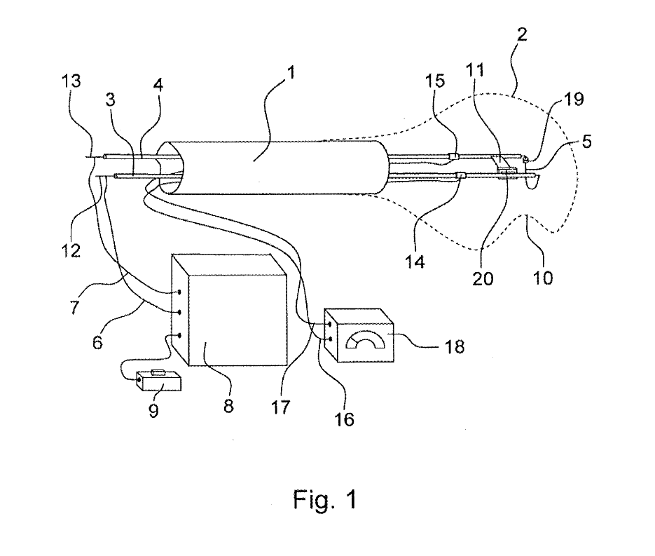

FIG. 1 shows a very schematic representation of the distal end region of a urological resectoscope with a working electrode, and

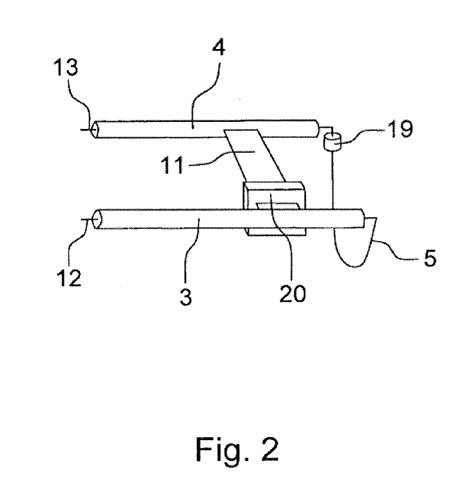

FIG. 2 shows an enlarged detail from FIG. 1 in the region of the working electrode.

FIG. 1 shows a perspective view of the distal end region of a urological instrument in the form of a urological resectoscope which substantially corresponds to the design of DE 10 2011 121 792 A1.

In FIG. 1, the distal end region of a shaft tube 1 of the resectoscope is illustrated, which in the example illustrated in FIG. 1 has been positioned in a bladder 2 indicated by a broken line. Two support arms 3 and 4, which are arranged parallel to one another and support a working electrode 5 between their distal ends, extend through the shaft tube 1. The working electrode consists of an uninsulated electrically conductive wire. The electrode arrangement 3, 4, 5 thus formed is connected via connecting cables 6, 7 to a high frequency generator 8 which is switchable by the usual foot switch 9.

In the conventional design for urological resectoscopes, the working electrode 5 is designed as a U-shaped loop. When a high frequency is applied to it, it cuts through tissue and can be used, for example, to remove a tumor 10. For this, with the high frequency generator 8 switched on the electrode arrangement must be moved by means of the support arms 3 and 4 in a suitable manner for cutting.

In the illustrated exemplary embodiment, a bipolar resectoscope is shown, that is to say, a resectoscope in which two electrodes are connected separately from one another to two poles of the high frequency generator 8, wherein between these electrodes current flows through the liquid in the bladder 2.

For this purpose, as shown in particular in FIG. 2 in an enlarged depiction of the distal end region of the working electrode 5, a counter-electrode 11, which is arranged between the support arms 3 and 4 with proximal spacing relative to the working electrode 5, is arranged in the distal end region of the working electrode 5.

The two support arms 3 and 4 are formed as rods made of insulating material or also as insulating tubes. A first electrical conductor 12, which in its distal end region projects beyond the support arm 3 and is formed there as the loop-shaped working electrode 5, extends in the interior of the support arm 3. As shown in particular in FIG. 2, the working electrode is connected at its other end by means of a first insulator 19 to the distal end of a second conductor 13 which runs through the support arm 4.

The counter-electrode 11 is connected conductively to the second conductor 13, but not to the first conductor 12, to which there is only an insulating connection by means of a second insulator 20. Thus the counter-electrode 11 is electrically connected only to the second conductor 13 and the working electrode 5 is electrically connected only to the first conductor 12.

Thus two electrodes 5 and 11, which are electrically insulated from one another and are separately connected by means of the conductors 12 and 13, are located in the distal end region of the electrode arrangement. As FIG. 1 shows, the conductors 12 and 13 are connected to the connecting cables 6 and 7 and run to separate poles of the high frequency generator 8.

If the high frequency generator 8 is switched on, different voltage poles are applied to the electrodes 5 and 11, and electrical current flows between them through the electrically conductive liquid in the bladder 2. Because of its high performance, this bipolar operating technique has recently become increasingly popular, but it also results in a very high energy input into the bladder 2.

Therefore, for protection of the patient's tissue in the region of the bladder 2, the temperature in the bladder should be monitored. A temperature measuring device is provided for this purpose.

The temperature measuring device according to the invention is illustrated in FIG. 1. It has two measuring electrodes 14 and 15. These are in each case arranged on one of the two support arms 3, 4, in the illustrated exemplary embodiment as simple sheet metal sleeves clamped onto the respective support arm. Measuring leads 16 and 17 lead to a resistance measuring device 18, by which the resistance between the measuring electrodes 14 and 15 is measured and displayed, and is set up remotely from the measuring electrodes outside the bladder 2.

In any case, in the region of the measuring electrodes 14 and 15, the support arms 3 and 4 are rigid and are held at a fixed distance from one another. The counter-electrode 11 also contributes to this. The support arms 3 and 4 and the counter-electrode 11 form a spacer, which maintains the spacing between the measuring electrodes 14 and 15.

In a customary manner, the resistance measuring device 18 can connect a current source and a measuring device, as well as the measuring path between the measuring electrodes 14 and 15 in series one after the other. This results in a measurement of the electrical resistance between the measuring electrodes 14 and 15 by means of the current through the liquid located between them.

Experiments have shown that the electrical conductivity, that is to say, the inverse of the electrical resistance, in a commercially available irrigation liquid with a physiological salt component, such as is usually used for irrigation in urological operations, for example in the temperature range from 20 to 45° Celsius, rises linearly from 15 to 25 mS/cm. Thus, conditions prevail here which are easily controllable and reproducible by means of measurement techniques.

### LIST OF REFERENCE SIGNS

1 shaft tube
2 bladder
3 support arm
4 support arm
5 working electrode
6 connecting cable
7 connecting cable
8 HF generator
9 foot switch
10 tumor
11 counter-electrode
12 first conductor
13 second conductor
14 measuring electrode
15 measuring electrode
16 measuring lead
17 measuring lead
18 resistance measuring device
19 first insulator
20 second insulator

1. A urological instrument having a working electrode, to which a high frequency can be applied in a bladder filled with liquid, and having a temperature sensor arranged at a fixed distance therefrom, characterized in that the temperature sensor has two measuring electrodes which are arranged at a fixed distance relative to one another, can be brought into liquid contact, and are connected to a resistance measuring device.

2. The urological instrument according to claim 1, wherein the measuring electrodes are fastened to an insulating spacer.

3. The urological instrument according to claim 1, wherein the measuring electrodes are connected by means of measuring leads to the remotely arranged resistance measuring device.

4. The urological instrument according to claim 1, wherein the measuring electrodes are rigidly connected to the working electrode.

5. The urological instrument according to claim 4, wherein the working electrode, which is held by two rigid support arms which are arranged parallel and spaced apart, wherein the support arms each support one of the measuring electrodes.

6. The urological instrument according to claim 2, wherein the measuring electrodes are connected by means of measuring leads to the remotely arranged resistance measuring device.

7. The urological instrument according to claim 2, wherein the measuring electrodes are rigidly connected to the working electrode.

8. The urological instrument according to claim 3, wherein the measuring electrodes are rigidly connected to the working electrode.

9. The urological instrument according to claim 6, wherein the measuring electrodes are rigidly connected to the working electrode.

10. The urological instrument according to claim 7, wherein the working electrode, which is held by two rigid support arms which are arranged parallel and spaced apart, wherein the support arms each support one of the measuring electrodes.

11. The urological instrument according to claim 8, wherein the working electrode, which is held by two rigid support arms which are arranged parallel and spaced apart, wherein the support arms each support one of the measuring electrodes.

12. The urological instrument according to claim 9, wherein the working electrode, which is held by two rigid support arms which are arranged parallel and spaced apart, wherein the support arms each support one of the measuring electrodes.
